(19) United States
(12) Patent Application Publication
(10) Pub. No.: US 2014/0024101 A1
(43) Pub. Date: Jan. 23, 2014

(54) METHOD FOR INCREASING THE REPLICATION CAPACITY OF AN INFLUENZA VIRUS IN CULTURED CELLS

(71) Applicants: Vincent Lotteau, Lyon (FR); Benoit De Chassey, Lyon (FR); Patrice Andre, Lyon (FR); Laurene Meyniel-Schicklin, Lyon (FR); Anne Aublin-Gex, Lyon (FR)

(72) Inventors: Vincent Lotteau, Lyon (FR); Benoit De Chassey, Lyon (FR); Patrice Andre, Lyon (FR); Laurene Meyniel-Schicklin, Lyon (FR); Anne Aublin-Gex, Lyon (FR)

(73) Assignees: Institut National de la Sante et de la Recherche Medicale (INSERM), Paris (FR); UNIVERSITÉ CLAUDE BERNARD - LYON 1, VILLEURBANNE CEDEX (FR)

(21) Appl. No.: 14/110,203

(22) PCT Filed: Apr. 10, 2012
(86) PCT No.: PCT/EP2012/056468

§ 371 Date: Oct. 7, 2013

(30) Foreign Application Priority Data
Apr. 8, 2011 (EP) 11305411.8

(51) Int. Cl. C12N 7/00

(52) U.S. Cl. CPC C12N 7/00
USPC 435/235.1

(57) ABSTRACT

The present invention relates to methods for increasing the replication capacity of an influenza virus in cultured cells. More particularly, the present invention relates to a method for increasing the replication capacity of an influenza virus in a cell comprising the steps consisting of i) infecting said cell with said influenza virus and ii) culturing said infected cell with a least one molecule selected from the group consisting of Dibucaine, Aprindine, Amiloride, Mevinolin, Simvastatin, Promathazine, Pranlukast, Nimodipine, Ibutilide hemifumarate Salt, Risperidone and derivatives or analogues thereof.

### FIELD OF THE INVENTION

The present invention relates to methods for increasing the replication capacity of an influenza virus in cultured cells.

### BACKGROUND OF THE INVENTION

Influenza viruses are one of the most ubiquitous viruses present in the world, affecting both humans and livestock. Influenza results in an economic burden, morbidity and even mortality, which are significant.

The influenza virus is an RNA enveloped virus with a particle size of about 125 nm in diameter. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and haemagglutinin (HA), which appear as spikes, 10 to 12 nm long, at the surface of the particles. It is these surface proteins, particularly the haemagglutinin that determine the antigenic specificity of the influenza subtypes. Virus strains are classified according to host species of origin, geographic site and year of isolation, serial number, and, for influenza A, by serological properties of subtypes of HA and NA. 16 HA subtypes (HI-HI6) and nine NA subtypes (N1-N9) have been identified for influenza A viruses. Viruses of all HA and NA subtypes have been recovered from aquatic birds, but only three HA subtypes (HI, H2, and H3) and two NA subtypes (N1 and N2) have established stable lineages in the human population since 1918. Only one subtype of HA and one of NA are recognised for influenza B viruses.

Influenza A viruses evolve and undergo antigenic variability continuously. A lack of effective proofreading by the viral RNA polymerase leads to a high rate of transcription errors that can result in amino-acid substitutions in surface glycoproteins. This is termed “antigenic drift”. The segmented viral genome allows for a second type of antigenic variation. If two influenza viruses simultaneously infect a host cell, genetic reassortment, called “antigenic shift” may generate a novel virus with new surface or internal proteins. These antigenic changes, both ‘drifts’ and ‘shifts’ are unpredictable and may have a dramatic impact from an immunological point of view as they eventually lead to the emergence of new influenza strains and that enable the virus to escape the immune system causing the well known, almost annual, epidemics. Both of these genetic modifications have caused new viral variants responsible for pandemic in humans.

HA is the most important antigen in defining the serological specificity of the different influenza strains. This 75-80 kD protein contains numerous antigenic determinants, several of which are in regions that undergo sequence changes in different strains (strain-specific determinants) and others in regions which are common to many HA molecules (common to determinants).

Influenza viruses cause epidemics almost every winter, with infection rates for type A or B virus as high as 40% over a six-week period. Influenza infection results in various disease states, from a sub-clinical infection through mild upper respiratory infection to a severe viral pneumonia. Typical influenza epidemics cause increases in incidence of pneumonia and lower respiratory disease as witnessed by increased rates of hospitalization or mortality. The severity of the disease is primarily determined by the age of the host, his immune status and the site of infection.

Elderly people, 65 years old and over, are especially vulnerable, accounting for 80-90% of all influenzarelated deaths in developed countries. Individuals with underlying chronic diseases are also most likely to experience such complications. Young infants also may suffer severe disease. These groups in particular therefore need to be protected. Besides these ‘at risk’-groups, the health authorities are also recommending to vaccinate healthy adults who are in contact with elderly persons.

Vaccination thus plays a critical role in controlling annual influenza epidemics. Currently available influenza vaccines are either inactivated or live attenuated influenza vaccine. Inactivated flu vaccines are composed of three possible forms of antigen preparation: inactivated whole virus, sub-virions where purified virus particles are disrupted with detergents or other reagents to solubilise the lipid envelope (so-called “split” vaccine) or purified HA and NA (subunit vaccine). These inactivated vaccines are given intramuscularly (i.m.) or intranasaly (i.n.).

Most human influenza vaccines are currently produced in embryonated hen's eggs. This production method benefits from decades of industrial experience, has consequently a good safety profile and is cost-effective. However, major drawbacks are associated with egg-based manufacturing of vaccine. Processes suffer from a limited capacity (one egg is approximately required to generate one vaccine dose), poor flexibility and restricted responsiveness, decreasing their ability to meet the demand in case of pandemics. Assuming that a sufficient quantity of eggs is available over the planned period, approximately 6-9 months might be needed for vaccine production. The low adaptability of the egg-based production process increases the risks of vaccine mismatch with circulating strains. These constraints therefore compromise the production of vaccines during an influenza pandemic, particularly if the strain is of avian origin (such as H5N1) and can not be produced in eggs. Within this context, it becomes critical to explore more robust alternative production methods.

Cell culture-based production systems offer a highly attractive alternative to egg-based processes. Mammalian cell culture is now considered an established technology for the production of therapeutic proteins or vaccines in the biopharmaceutical industry. Production is operated within a closed and controlled environment, and can be readily transferred to industrial manufacturing scales. The risks for microbiological contamination are significantly reduced and allergic reactions induced by egg proteins are absent. Furthermore, it is expected that the cell culture produced vaccines are more similar to the primary human isolate than egg-adapted viruses, inducing a higher cross-reactive protective immune response.

Several mammalian cell lines such as Madin Darbin Canine Kidney (MDCK) (G. F. Rimmelzwaan, M. Baars, E. C. J. Claas and A. D. M. E. Osterhaus, Comparison of RNA hybridization, hemagglutination assay, titration of infectious virus and immunofluorescence as methods for monitoring influenza virus replication in vitro, J Virol Methods 74 (1998), pp. 57-66.; J. T. M. Voeten, R. Brands, A. M. Palache, G. J. M. van Scharrenburg, G. F. Rimmelzwaan and A. D. M. E. Osterhaus et al., Chracterization of high-growth reassortant influenza A viruses generated in MDCK cells cultured in serum-free medium, Vaccine 17 (1999), pp. 1942-1950.), human embryonic retinal cells (PER.C6) (M. G. Pau, C. Ophorst, M. H. Koldijk, G. Schouten, M. Mehtali and F. Uytdehaag, The human cell line PER.C6 provides a new manufacturing system for the production of influenza vaccines, Vaccine 19 (2001), pp. 2716-2721.), HEK-293 or monkey kidney cells (Vero) (O. Kistner, P. N. Barrett, W. Mundt, M. Reiter, S. Schober-Bendixen and F. Dorner, Development of a mammalian cell (Vero) derived candidate influenza virus vaccine, Vaccine 16 (9-10) (1998), pp. 960-968.; R. Youil, Q. Su, T. J. Toner, C. Szymkowiak, W. S. Kwan and B. Rubin et al., Comparative study of influenza virus replication in Vero and MDCK cell lines, J Virol Methods 120 (2004), pp. 23-31.) have been explored for the production of influenza particles. Recently, it was also suggested to use HEK-293 cell line for the production of influenza vaccines (Le Ru A, Jacob D, Transfiguracion J, Ansorge S, Henry O, Kamen A A. Scalable production of influenza virus in HEK-293 cells for efficient vaccine manufacturing. Vaccine. 2010 May 7; 28(21):3661-71. Epub 2010 Mar. 26.) However production of influenza vaccine in mammalian cell lines and especially in human cell lines does not currently allow infectious particles at levels comparable with eggs.

In light of the foregoing, a need in the art exists for method that will allow the production of infectious particles with high yield for the production of influenza vaccines. Typically, compound that will allow increasing the replication of influenza virus in cultured cells are particularly highly desirable.

### SUMMARY OF THE INVENTION

The present invention relates to methods for increasing the replication capacity of an influenza virus in cultured cells. More particularly, the present invention relates to a method for increasing the replication capacity of an influenza virus in a cell comprising the steps consisting of i) infecting said cell with said influenza virus and ii) culturing said infected cell with a least one molecule selected from the group consisting of Dibucaine, Aprindine, Amiloride, Mevinolin, Simvastatin, Promathazine, Pranlukast, Nimodipine, Ibutilide hemifumarate Salt, Risperidone and derivatives or analogues thereof.

### DETAILED DESCRIPTION OF THE INVENTION

The inventors have now identified different FDA approved molecules that increase the replication of influenza virus in cultured cells, and that can be used in cell culture-based influenza vaccine production. Said molecules are depicted in Table 1 and are known per se by the skilled man in the art:

| | DrugBank | |
|---|---|---|
| Name | id | IUPAC name |
| Dibucaine | DB00527 | 2-butoxy-N-[2-(diethylamino)ethyl]quinoline-4-carboxamide |
| amiloride | DB00594 | 3,5-diamino-6-chloro-N-(diaminomethylidene)pyrazine-2- |
| | | carboxamide |
| Aprindine | DB01429 | {3-[(2,3-dihydro-1H-inden-2- |
| | | yl)(phenyl)amino]propyl}diethylamine |
| Pranlukast | DB01411 | N-[4-oxo-2-(2H-1,2,3,4-tetrazol-5-yl)-4H-chromen-7-yl]-4-(4- |
| | | phenylbutoxy)benzamide |
| Promethazine | DB01069 | dimethyl[1-(10H-phenothiazin-10-yl)propan-2-yl]amine |
| Simvastatin | DB00641 | (1S,3R,7S,8S,8aR)-8-{2-[(4R)-4-hydroxy-6-oxooxan-2- |
| | | yl]ethyl}-3,7-dimethyl-1,2,3,7,8,8a-hexahydronaphthalen-1-yl |
| | | 2,2-dimethylbutanoate |
| Mevinolin | DB00227 | (1S,3R,7S,8S,8aR)-8-{2-[(2R,4R)-4-hydroxy-6-oxooxan-2- |
| | | yl]ethyl}-3,7-dimethyl-1,2,3,7,8,8a-hexahydronaphthalen-1-yl |
| | | (2S)-2-methylbutanoate |
| Nimodipine | DB00393 | 3-(2-methoxyethyl) 5-propan-2-yl 2,6-dimethyl-4-(3- |
| | | nitrophenyl)-1,4-dihydropyridine-3,5-dicarboxylate |
| Risperidone | DB00734 | 3-{2-[4-(6-fluoro-1,2-benzoxazol-3-yl)piperidin-1-yl]ethyl}-2- |
| | | methyl-4H,6H,7H,8H,9H-pyrido[1,2-a]pyrimidin-4-one |
| ibutilide | DB00308 | N-(4-{4-[ethyl(heptyl)amino]-1- |
| hemifumarate | | hydroxybutyl}phenyl)methanesulfonamide |
| salt | | |

Accordingly, the present invention relates to a method for increasing the replication capacity of an influenza virus in a cell comprising the steps consisting of i) infecting said cell with said influenza virus and ii) culturing said infected cell with a least one molecule selected from the group consisting of Dibucaine, Aprindine, Amiloride, Mevinolin, Simvastatin, Promathazine, Pranlukast, Nimodipine, Ibutilide hemifumarate Salt, Risperidone and derivatives or analogues thereof.

According to the present invention, any influenza virus strain can be used. Preferably, said influenza virus strain corresponds to a clinical isolate of at least one circulating strain of an influenza A or B virus. For the production of a safe and effective vaccine it is indeed important that the selected influenza virus strains are closely related to the circulating strains. Type A viruses are principally classified into antigenic sub-types on the basis of two viral surface glycoproteins, hemagglutinin (HA) and neuraminidase (NA). There are currently 16 identified HA sub-types (designated H1 through H16) and 9 NA sub-types (N1 through N9) all of which can be found in wild aquatic birds. Of the 135 possible combinations of HA and NA, only four (H1N1, H1N2, H2N2, H5N1 and H3N2) have widely circulated in the human population since the virus was first isolated in 1933.

In a particular embodiment, the clinical isolate can be made into a high growth strain by reassortment with a high growth master donor strain, or by multiple passages of the clinical isolate in continuous mammalian cell lines, with selection of high growth variants. The clinical isolates are preferably reassorted with laboratory high growth master donor strains in culture, and the reassortants selected that have HA and NA genes from the isolates, and internal genes from the high growth master laboratory strains. For example, the resulting strain for the influenza A component can be a reassortant virus that contains internal genes from the master donor strain A/PR/8/34 (H1N1), which provides high growth in cells, as well as at least the HA gene coding for at least one surface antigen of the clinical isolate of the influenza virus (using known methods, e.g., according to Robertson et al., Biologicals 20:213-220 (1992)). Such reassortants can be made more rapidly than high growth strains made by multiple passages of the clinical isolates.

In a further preferred embodiment, the infection of the cells with influenza viruses is carried out at an m.o.i. (multiplicity of infection) of about 0.0001 to 10, preferably of 0.002 to 0.5.

The term “increased replication capacity,” as used herein with reference to a viral phenotype, means that the virus grows to a greater titer in the presence of a molecule as above described relative to parent virus grown in the absence of said molecule. In one embodiment, the presence of said molecule which will increase the ability of an influenza virus to replicate in a cell by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to said influenza virus cultured in the absence of said molecule.

According to the invention, any eukaryotic cell may be used. Preferably said cell is a mammalian cell. Typically said mammalian cells include but are not limited to cells from humans, dogs, cats, cattle, horses, sheep, pigs, goats, and rabbits. In a particular embodiment the cell is a human cell. In another particular embodiment said cell is a cell line. Typically the cell is certified according to the WHO requirements for vaccine production. The requirements for certifying such cell lines include characterization with respect to at least one of genealogy, growth characteristics, immunological markers, virus susceptibility tumorigenicity and storage conditions, as well as by testing in animals, eggs, and cell culture. Non-limiting examples of cell lines that can be suitable for the invention include but are not limited to BS-C-1, CV-1, Vero, Vero 76, Vero C1008, Vero 76, Cos-1, Cos-7, FR11K-4, LLC-MK2 original, LLC-MK2 derivative, MDCK, RD, A549, MRC-5, KB, PER.C6, HEK-293 and CaCo-2 cells. It is preferred to establish a complete characterization of the cell line to be used. Data that can be used for the characterization of a cell line to be used in the present invention includes (a) information on its origin, derivation, and passage history; (b) information on its growth and morphological characteristics; (c) distinguishing features, such as biochemical, immunological, and cytogenetic patterns which allow the cells to be clearly recognized among other cell lines; and (d) results of tests for tumorigenicity. Preferably, the passage level, or population doubling, of the cell line used is as low as possible.

Typically, cells are cultured in a standard commercial culture medium, such as Dulbecco's modified Eagle's medium supplemented with serum (e.g., 10% fetal bovine serum), or in serum free medium, under controlled humidity and C02 concentration suitable for maintaining neutral buffered pH (e.g., at pH between 7.0 and 7.2). Suitable serum free media are described, for example, in U.S. Provisional Application No. 60/638,166, filed Dec. 23, 2004, and in U.S. Provisional Application No. 60/641,139, filed Jan. 5, 2005, each of which is hereby incorporated by reference in its entirety. Optionally, the medium contains antibiotics to prevent bacterial growth, e.g., penicillin, streptomycin, etc., and/or additional nutrients, such as L-glutamine, sodium pyruvate, nonessential amino acids, additional supplements to promote favorable growth characteristics, e.g., trypsin, (3-mercaptoethanol, and the like.

Cells for production of influenza virus can be cultured in serum-containing or serum free medium. In some case, e.g., for the preparation of purified viruses, it is desirable to grow the cells in serum free conditions. Cells can be cultured in small scale, e.g., less than 25 ml medium, culture tubes or flasks or in large flasks with agitation, in rotator bottles, or on microcarrier beads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor) in flasks, bottles or reactor cultures. Microcarrier beads are small spheres (in the range of 100-200 microns in diameter) that provide a large surface area for adherent cell growth per volume of cell culture. For example a single liter of medium can include more than 20 million microcarrier beads providing greater than 8000 square centimeters of growth surface. For commercial production of viruses, e.g., for vaccine production, it is often desirable to culture the cells in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany).

Typically, the molecule of the present invention is added to a final concentration of 1 nM to 1 mM.

Combinations of said molecules are also possible.

Accordingly a further aspect of the invention relates to a culture medium suitable for increasing the replication of an influenza virus in a cell culture comprising an amount of at least one molecule selected form Dibucaine, Aprindine, Amiloride, Mevinolin, Simvastatin, Promathazine, Pranlukast, Nimodipine, Ibutilide hemifumarate Salt, Risperidone and derivatives or analogues thereof.

The cells can be grown in culture under conditions permissive for replication and assembly of viruses. In embodiments, cells can be cultured at a temperature below about 37° C., preferably at a temperature equal to, or less than, about 35° C. Typically, the cells are cultured at a temperature between about 32° C. and about 35° C. In some embodiments, the cells are cultured at a temperature between about 32° C. and 34° C., e.g., at about 33° C.

The culturing of the cells is carried out as a rule at a regulated pH which is preferably in the range from pH 6.6 to pH 7.8, in particular in the range from pH 6.8 to pH 7.3.

Furthermore, the pO2 value can advantageously be regulated and is then as a rule between 25% and 95%, in particular between 35% and 60% (based on the air saturation).

In a particular embodiment, a protease is added to the culture medium of the cells. The addition of the protease which brings about the cleavage of the precursor protein of hemagglutinin and thus the adsorption of the viruses on the cells, can be carried out according to the invention shortly before, simultaneously to or shortly after the infection of the cells with influenza viruses. If the addition is carried out simultaneously to the infection, the protease can either be added directly to the cell culture to be infected or, for example, as a concentrate together with the virus inoculate. The protease is preferably a serine protease, and particularly preferably trypsin. Typically, trypsin may be added to the cell culture to a final concentration of 1 to 200 μg/ml, preferably 5 to 50 μg/ml, and particularly preferably 5 to 30 μg/ml in the culture medium.

Following culture for a suitable period of time to permit replication of the virus to high titer, the virus can be recovered. Viruses can typically be recovered from the culture medium, in which infected (transfected) cells have been grown. Typically crude medium is clarified prior to concentration of influenza viruses. Common methods include filtration, ultrafiltration, adsorption on barium sulfate and elution, and centrifugation. For example, crude medium from infected cultures can first be clarified by centrifugation at, e.g., 1000-2000×g for a time sufficient to remove cell debris and other large particulate matter, e.g., between 10 and 30 minutes. Alternatively, the medium is filtered through a 0.8 um cellulose acetate filter to remove intact cells and other large particulate matter. Optionally, the clarified medium supernatant is then centrifuged to pellet the influenza viruses, e.g., at 15,000×g, for approximately 3-5 hours. Following resuspension of the virus pellet in an appropriate buffer, such as STE (0.01 MTris-HCl; 0.15MNaCl; 0.0001 MEDTA) or phosphate buffered saline (PBS) at pH 7.4, the virus is concentrated by density gradient centrifugation on sucrose (60% 12%) or potassium tartrate (50%-10%). Either continuous or step gradients, e.g., a sucrose gradient between 12% and 60% in four 12% steps, are suitable. The gradients are centrifuged at a speed, and for a time, sufficient for the viruses to concentrate into a visible band for recovery. Alternatively, and for most large scale commercial applications, virus is elutriated from density gradients using a zonal-centrifuge rotor operating in continuous mode. Additional details sufficient to guide one of skill through the preparation of influenza viruses from tissue culture are provided, e.g., in Furminger. Vaccine Production, in Nicholson et al. (eds) Textbook of Influenza pp. 324-332; Merten et al. (1996) Production of influenza virus in cell cultures for vaccine preparation, in Cohen & Shafferman (eds) Novel Strategies in Design and Production of Vaccines pp. 141-151, and U.S. Pat. No. 5,690,937, U.S. publication application nos. 20040265987, 20050266026 and 20050158342, which are incorporated by reference herein. If desired, the recovered viruses can be stored at −80° C. in the presence of sucrose-phosphate-glutamate (SPG) as a stabilizer.

The method of the present invention is particularly useful for the production of influenza virus vaccines.

The resulting replicated virus can be indeed concentrated as above described and then be inactivated or attenuated using any method well known in the art.

Inactivated influenza virus vaccines of the invention are typically provided by inactivating replicated virus of the invention using known methods, such as, but not limited to, formalin or .beta.-propiolactone treatment. Inactivated vaccine types that can be used in the invention can include whole-virus (WV) vaccine or subvirion (SV) virus vaccine. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen vaccines. In general, the responses to SV and surface antigen (i.e., purified HA or NA) vaccines are similar. An experimental inactivated WV vaccine containing an NA antigen immunologically related to the epidemic virus and an unrelated HA appears to be less effective than conventional vaccines. Inactivated vaccines containing both relevant surface antigens are preferred.

Live, attenuated influenza virus vaccines, using replicated virus of the invention, can also be used for preventing or treating influenza virus infection, according to known method steps: Attenuation is preferably achieved in a single step by transfer of attenuating genes from an attenuated donor virus to a replicated isolate or reassorted virus according to known methods (see, e.g., Murphy, Infect. Dis. Clin. Pract. 2:174-181 (1993)). Since resistance to influenza A virus is mediated by the development of an immune response to the HA and NA glycoproteins, the genes coding for these surface antigens must come from the reassorted viruses or high growth clinical isolates. The attenuating genes are derived from the attenuated parent. In this approach, genes that confer attenuation preferably do not code for the HA and NA glycoproteins. Otherwise, these genes could not be transferred to reassortants bearing the surface antigens of the clinical virus isolate.

Many donor viruses have been evaluated for their ability to reproducibly attenuate influenza viruses. As a non-limiting example, the A/Ann Arbor(AA)/6/60 (H2N2) cold adapted (ca) donor virus can be used for attenuated vaccine production (see, e.g., Edwards, J. Infect. Dis. 169:68-76 (1994); Murphy, Infect. Dis. Clin. Pract. 2:174-181 (1993)). Additionally, live, attenuated reassortant virus vaccines can be generated by mating the donor virus with a virulent replicated virus of the invention. Reassortant progeny are then selected at 25° C. (restrictive for replication of virulent virus), in the presence of an H2N2 antiserum, which inhibits replication of the viruses bearing the surface antigens of the attenuated A/AA/6/60 (H2N2) ca donor virus.

A large series of H1N1 and H3N2 reassortants have been evaluated in humans and found to be satisfactorily: (a) infectious, (b) attenuated for seronegative children and immunologically primed adults, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible adults and children.

Other attenuating mutations can be introduced into influenza virus genes by site-directed mutagenesis to rescue infectious viruses bearing these mutant genes. Attenuating mutations can be introduced into non-coding regions of the genome, as-well as into coding regions. Such attenuating mutations can also be introduced into genes other than the HA or NA, e.g., the PB2 polymerase gene (Subbarao et al., J. Virol. 67:7223-7228 (1993)). Thus, new donor viruses can also be generated bearing attenuating mutations introduced by site-directed mutagenesis, and such new donor viruses can be used in the production of live attenuated reassortants H1N1 and H3N2 vaccine candidates in a manner analogous to that described above for the A/AA/6/60 ca donor virus. Similarly, other known and suitable attenuated donor strains can be reassorted with replicated influenza virus of the invention to obtain attenuated vaccines suitable for use in the vaccination of mammals. (Ewami et al., Proc. Natl. Acad. Sci. USA 87:3802-3805 (1990); Muster et al., Proc. Natl. Acad. Sci. USA 88:5177-5181 (1991); Subbarao et al., J. Virol. 67:7223-7228 (1993); U.S. patent application Ser. No. 08/471,100, which references are entirely incorporated by reference)

It is preferred that such attenuated viruses maintain the genes from the replicated virus that encode antigenic determinants substantially similar to those of the original clinical isolates. This is because the purpose of the attenuated vaccine is to provide substantially the same antigenicity as the original clinical isolate of the virus, while at the same time lacking infectivity to the degree that the vaccine causes minimal chance of inducing a serious pathogenic condition in the vaccinated mammal.

The replicated virus that is attenuated or inactivated may be then formulated in a vaccine composition.

Vaccine compositions of the present invention, suitable for inoculation or for parenteral or oral administration, comprise attenuated or inactivated influenza viruses, optionally further comprising sterile aqueous or non-aqueous solutions, suspensions, and emulsions. The composition can further comprise auxiliary agents or excipients, as known in the art.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are entirely incorporated herein by reference, included all references cited therein.

When a vaccine composition of the present invention is used for administration to an individual, it can further comprise salts, buffers, adjuvants, or other substances which are desirable for improving the efficacy of the composition.

Adjuvants are substances that can be used to augment a specific immune response. Normally, the adjuvant and the composition are mixed prior to presentation to the immune system, or presented separately, but into the same site of the mammal being immunized.

Heterogeneity in the vaccine may be provided by mixing replicated influenza viruses for at least two influenza virus strains, such as 2-50 strains or any range or value therein. Influenza A or B virus strains having a modem antigenic composition are preferred. According to the present invention, vaccines can be provided for variations in a single strain of an influenza virus or for more than one strain of influenza viruses, using techniques known in the art.

Once prepared the vaccine composition may be then administered in a subject in need thereof. Typically, an attenuated or inactivated vaccine composition of the present invention may thus be provided either before the onset of infection (so as to prevent or attenuate an anticipated infection) or after the initiation of an actual infection. For example, administration of such a vaccine composition may be by various parenteral routes such as subcutaneous, intravenous, intradermal, intramuscular, intraperitoneal, intranasal, oral or transdermal routes. Parenteral administration can be by bolus injection or by gradual perfusion over time. A preferred mode of using a vaccine composition of the present invention is by intramuscular or subcutaneous application. See, e.g., Berkow, infra, Goodman, infra, Avery, infra and Katzung, infra, which are entirely incorporated herein by reference, including all references cited therein.

The vaccine composition is administered to the subject in a effective amount. According to the present invention, an “effective amount” of a vaccine composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent preferred dose ranges. However, the most preferred dosage will be tailored to the individual subject, as is understood and determinable by one of skill in the art, without undue experimentation.

The invention will be further illustrated by the following figures and examples. However, these examples and figures should not be interpreted in any way as limiting the scope of the present invention.

### FIGURES

FIG. 1: MDCK or A549 cells were treated with increasing concentrations of Dibucaine (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 2: MDCK or A549 cells were treated with increasing concentrations of Amiloride (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 3: MDCK or A549 cells were treated with increasing concentrations of Aprindine (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 4: MDCK or A549 cells were treated with increasing concentrations of Pranlukast (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 5: MDCK or A549 cells were treated with increasing concentrations of Promethazine (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 6: MDCK or A549 cells were treated with increasing concentrations of Simvastatin (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 7: MDCK or A549 cells were treated with increasing concentrations of Mevinolin (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 8: MDCK or A549 cells were treated with increasing concentrations of Nimodipine (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 9: MDCK or A549 cells were treated with increasing concentrations of Risperidone (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

FIG. 10: MDCK or A549 cells were treated with increasing concentrations of Ibutilide Hemifumarate salt (Δ) or DMSO (♦) immediately before infection with H1N1 (respectively MOI 0.01 or MOI 0.1), H3N2 (MOI 0.6) or H5N1 (respectively MOI 0.001 or MOI 0.01). 24 h and 48 h post infection, supernatants were harvested and tested for the neuraminidase activity using a fluorometric assay. Fluorescence curves are given showing the effect of molecules on viral replication.

### EXAMPLE

Material & Methods

Cells and Virus

The A549 human lung epithelial cells line and the Madin-Darby canine kidney cells (ECACC,) were grown in DMEM media (GibCo, 41966052) supplemented with 100 U.ml penicilline/streptomycin (GibCo, 15140130) and 10% fetal calf serum (PAN, 3302-P221126) at 37° C. and 5% CO2.

The epidemic A/H1N1/New Caledonia/P10, A/H3N2/Wyoming and A/H5N1/Vietnam strains were propagated in MDCK cells in DMEM supplemented with 1 μg.ml⁻¹ modified trypsin TPCK (Sigma, T3053) in absence of FCS. Virus stocks were titrated by standard plaque assay on MDCK cells using an agar overlay medium.

Molecules

All the molecules were solubilized in DMSO at a stock concentration of 20 mM.

Virus Infection

Cells (MDCK or A549) were washed twice with D-PBS 1× (GibCo, 14190). Molecules were added at indicated concentrations. MDCK and A549 cells were then infected with H1N1 (respectively MOI 0.01 and 0.1), with H3N2 (MOI 0.6) or with H5N1 (respectively MOI 0.001 and 0.01) in DMEM supplemented with 0.2 μg.ml⁻¹ trypsin TPCK (infection medium) and incubated for 24 h or 48 h in infection medium at 37° C. and 5% CO₂.

Titer Measure by Neuraminidase Activity

Influenza virus neuraminidase is able to cleave the methyl-umbelliferyl-N-acetylneuraminic acid (4-MUNANA, Sigma M8639) modifying its emission wavelength in a dose-dependent manner.

In 96-black plate (Corning, 3631), 25 μl infection supernatants were diluted in 25 μl D-PBS1× containing calcium and magnesium (GibCo, 14040) and 50 μl of 20 μM 4-MUNANA. After 1 h incubation at 37° C., 100 μl of glycine 0.1M 25% ethanol pH10.7 was added. Measures were done with TECAN infinite M1000 instrument at 365 nm excitation wavelength and 450 nm emission wavelength.

Results

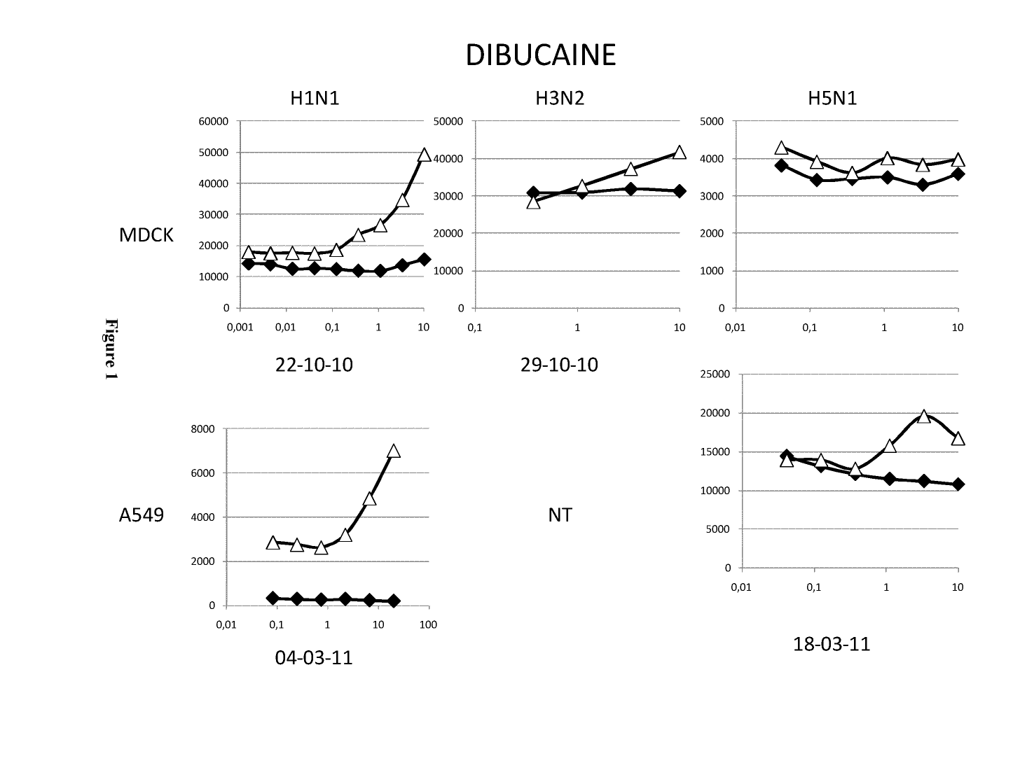

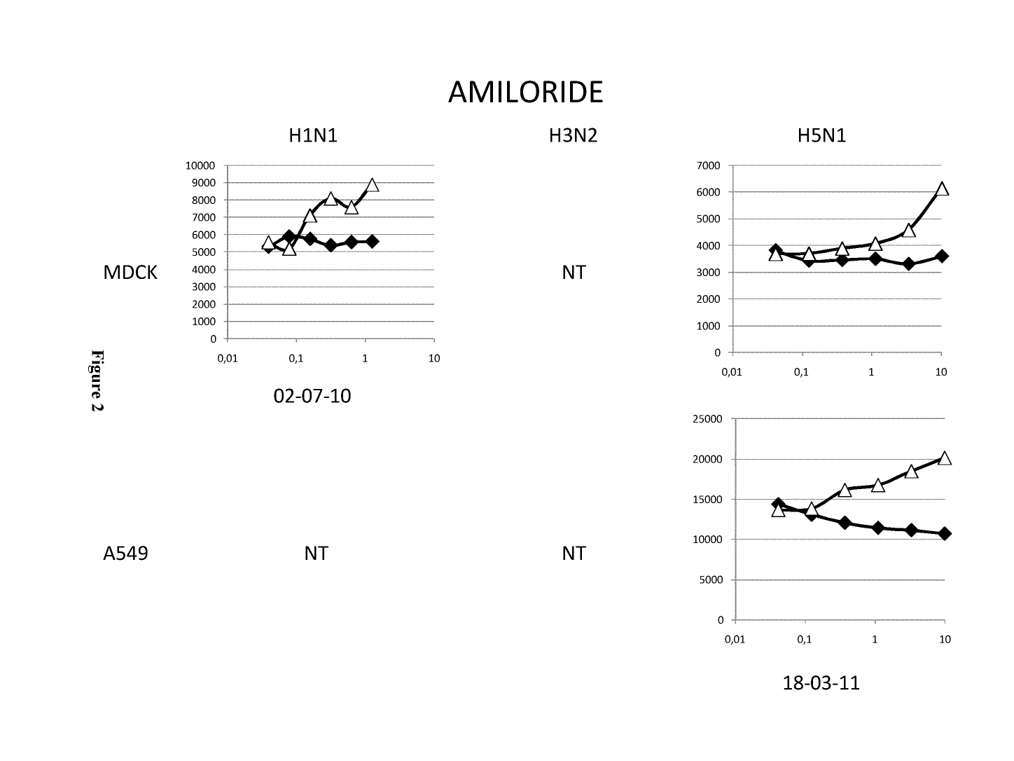

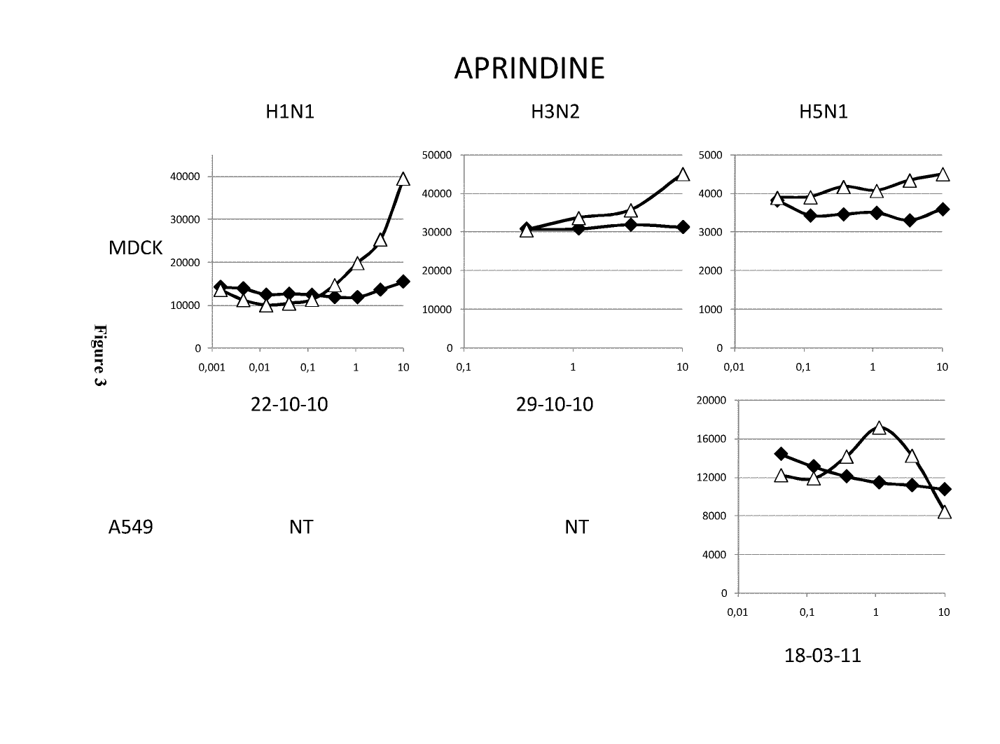

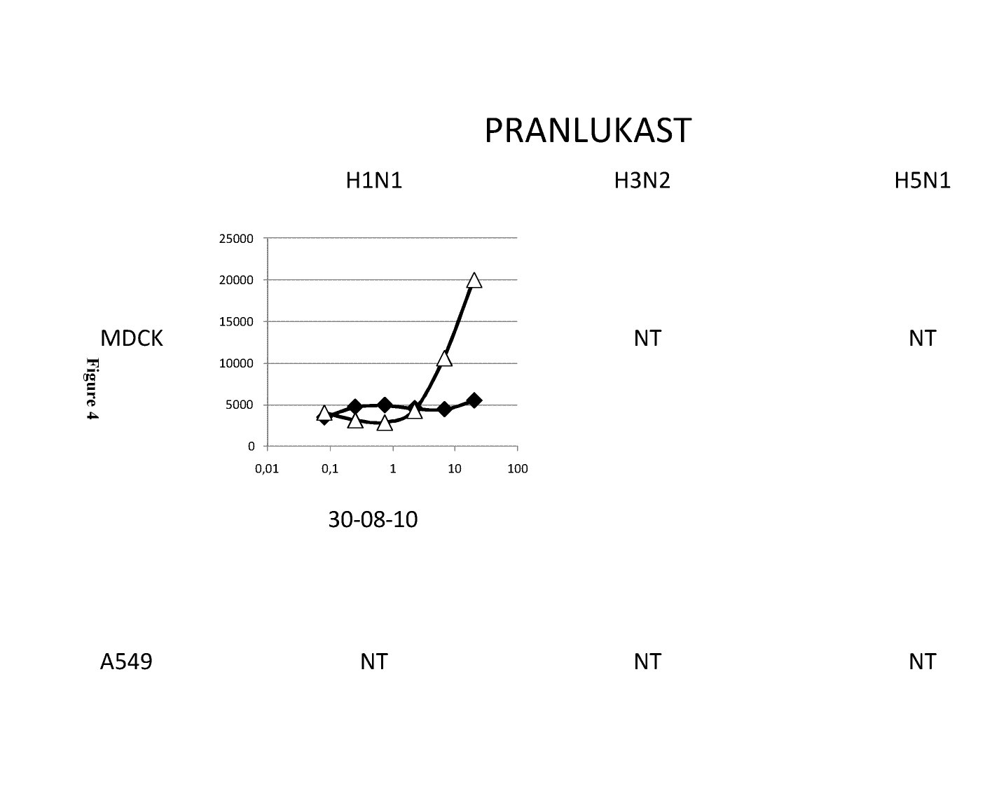

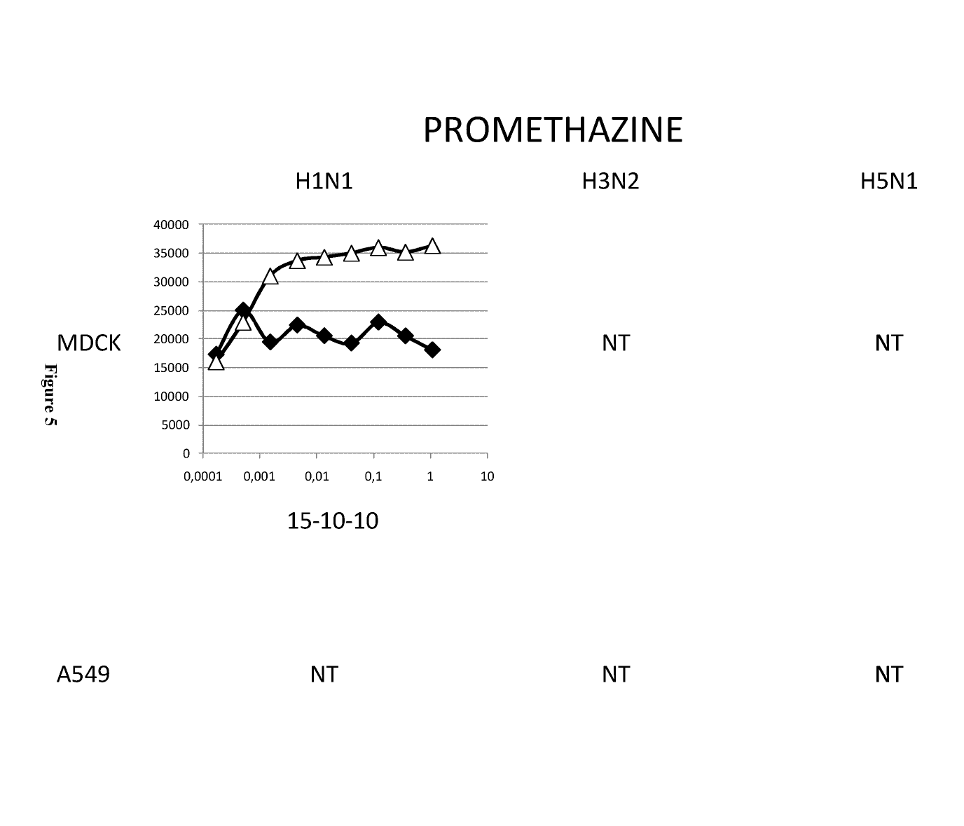

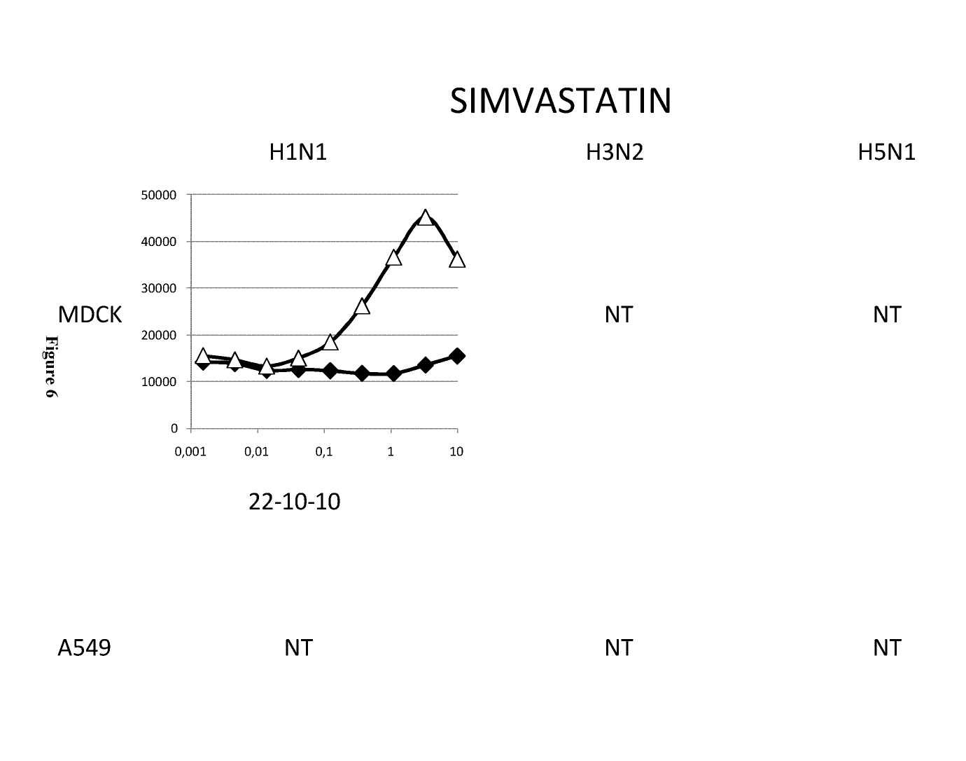

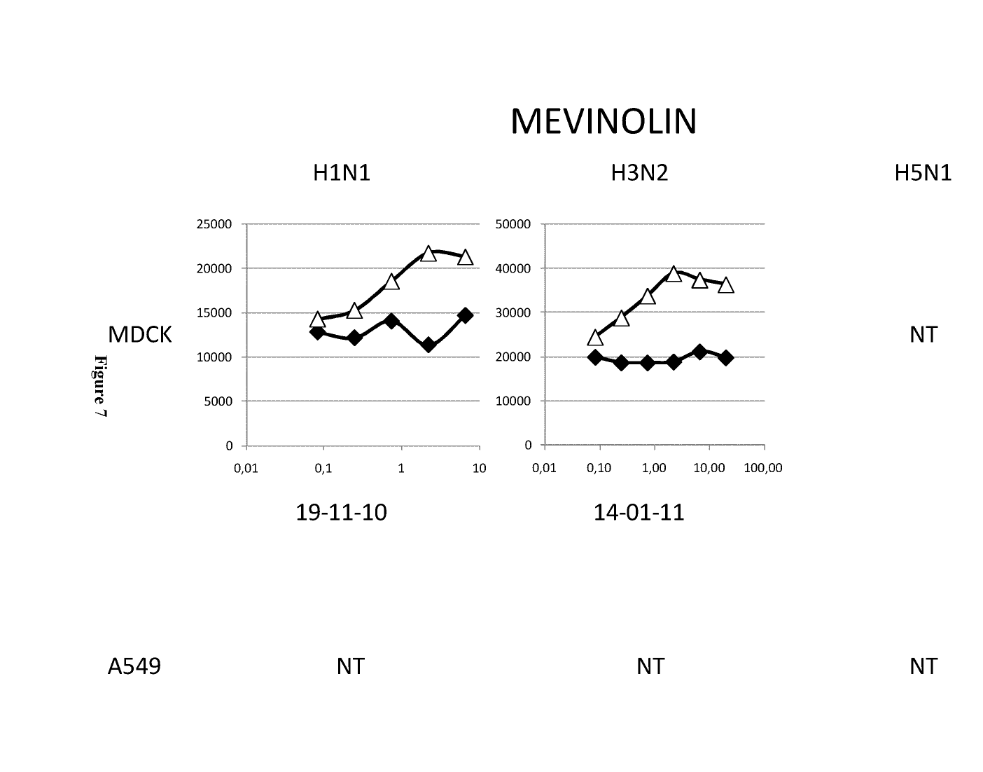

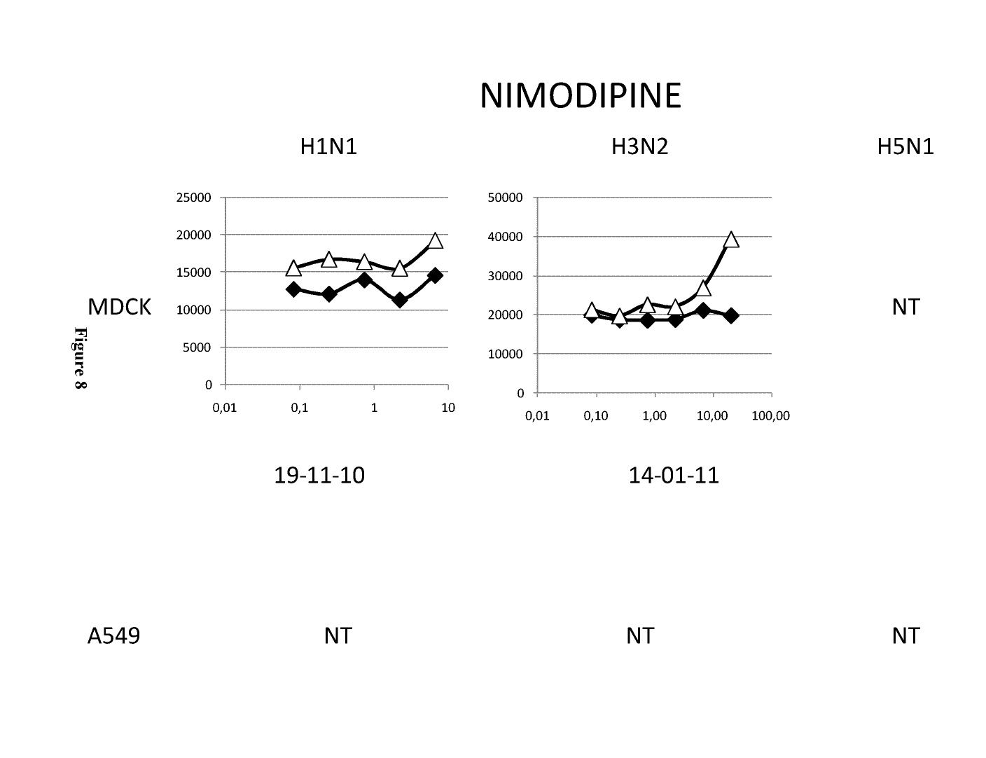

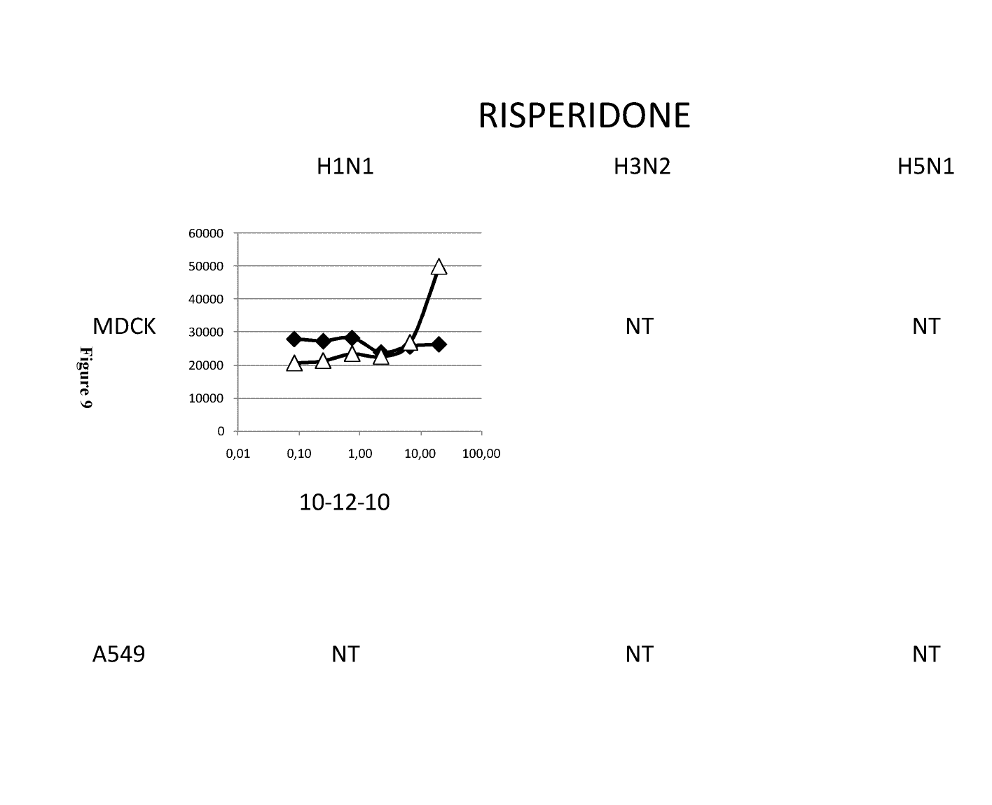

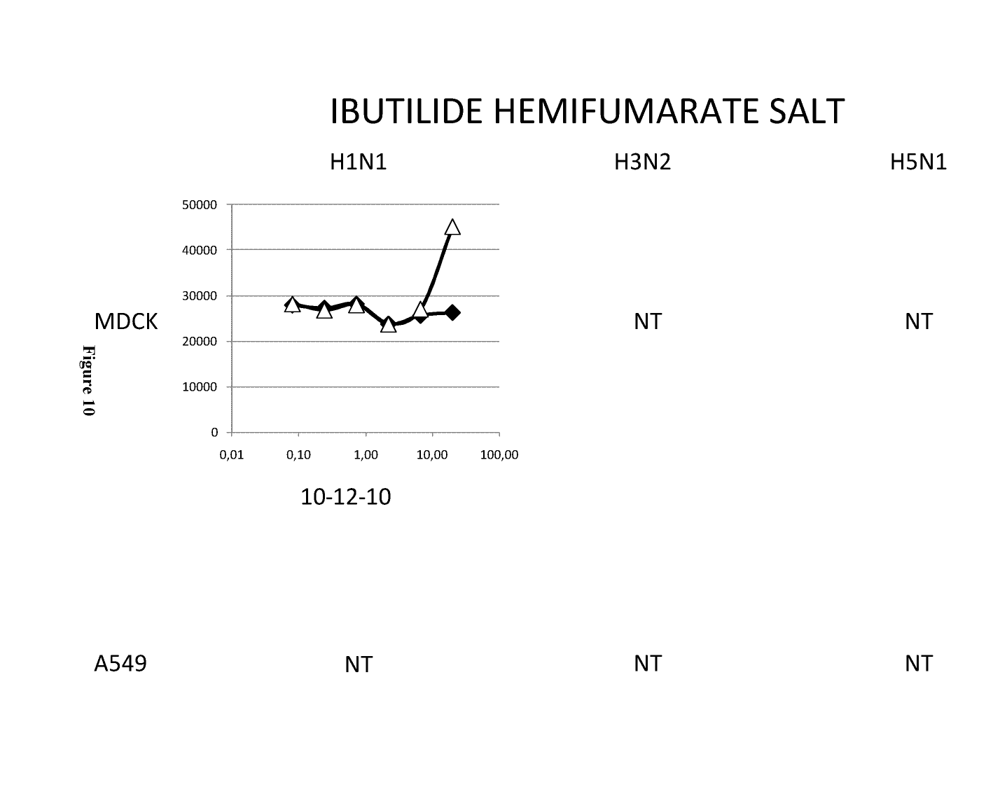

All the results are depicted in FIGS. 1-10.

### REFERENCES

Throughout this application, various references describe the state of the art to which this invention pertains. The disclosures of these references are hereby incorporated by reference into the present disclosure.

1. A method for increasing the replication capacity of an influenza virus in a cell comprising the steps of i) infecting said cell with said influenza virus and ii) culturing said infected cell with a least one molecule selected from the group consisting of Aprindine, Dibucaine, Amiloride, Mevinolin, Simvastatin, Promathazine, Pranlukast, Nimodipine, Ibutilide hemifumarate Salt, Risperidone and derivatives or analogues thereof.

2. The method according to claim 1 wherein said influenza virus is a circulating strain of an influenza A or B virus.

3. The method according to claim 2 wherein said influenza virus is selected from the group consisting of H1N1, H1N2, H2N2, H5N1 and H3N2 viruses.

4. The method according to claim 1 wherein said cell is a mammalian cell.

5. The method according to claim 4 wherein said cell is from a cell line selected from the group consisting of BS-C-1, CV-1, Vero, Vero 76, Vero C1008, Vero 76, Cos-1, Cos-7, FR11K-4, LLC-MK2 original, LLC-MK2 derivative, MDCK, RD, A549, MRC-5, KB, PER.C6, HEK-293 and CaCo-2 cells.

6. (canceled)

7. A culture medium suitable for increasing the replication of an influenza virus in a cell culture comprising an amount of at least one molecule selected from the group consisting of Aprindine, Dibucaine, Amiloride, Mevinolin, Simvastatin, Promathazine, Pranlukast, Nimodipine, Ibutilide hemifumarate Salt, Risperidone and derivatives or analogues thereof.

8. A method of producing influenza virus, comprising the steps of
i) infecting a cell with said influenza virus and
ii) culturing said infected cell with a least one molecule selected from the group consisting of Aprindine, Dibucaine, Amiloride, Mevinolin, Simvastatin, Promathazine, Pranlukast, Nimodipine, Ibutilide hemifumarate Salt, Risperidone and derivatives or analogues thereof.

9. The method according to claim 8 wherein said influenza virus is a circulating strain of an influenza A or B virus.

10. The method according to claim 9 wherein said influenza virus is selected from the group consisting of H1N1, H1N2, H2N2, H5N1 and H3N2 viruses.

11. The method according to claim 8 wherein said cell is a mammalian cell.

12. The method according to claim 4 wherein said cell is from a cell line selected from the group consisting of BS-C-1, CV-1, Vero, Vero 76, Vero C1008, Vero 76, Cos-1, Cos-7, FR11K-4, LLC-MK2 original, LLC-MK2 derivative, MDCK, RD, A549, MRC-5, KB, PER.C6, HEK-293 and CaCo-2 cells.
